(19) United States
(12) Patent Application Publication
(10) Pub. No.: US 2024/0144840 A1
(43) Pub. Date: May 2, 2024

(54) INFORMATION PROCESSING METHOD, INFORMATION PROCESSING DEVICE, AND NON-TRANSITORY COMPUTER READABLE RECORDING MEDIUM

(71) Applicant: Panasonic Intellectual Property Corporation of America, Torrance, CA (US)

(72) Inventors: Kazunobu KONISHI, Osaka (JP); Taro SUZUKI, Ibaraki (JP); Masafumi ISHIKAWA, Tokyo (JP); Hiroko IZUMI, Kanagawa (JP)

(73) Assignee: Panasonic Intellectual Property Corporation of America, Torrance, CA (US)

(21) Appl. No.: 18/407,098

(22) Filed: Jan. 8, 2024

Related U.S. Application Data
(63) Continuation of application No. PCT/JP2022/021650, filed on May 26, 2022.

(30) Foreign Application Priority Data
Jul. 12, 2021 (JP) 2021-115098

(51) Int. Cl. G09B 19/00; A61B 5/00; A61B 5/11

(52) U.S. Cl. CPC G09B 19/003; A61B 5/1114; A61B 5/6828; A61B 2562/0219

(57) ABSTRACT

A server acquires, on the basis of sensing data about a person, lifting information indicating a lifting state of a leg of the person, acquires height information indicating a height of a step from a floor in a space where the person moves, determines, on the basis of the lifting information and the height information, a dangerous level of a walk of the person, generates walk assist information in accordance with the dangerous level, and outputs the walk assist information.

### TECHNICAL FIELD

This disclosure relates to a technology of assisting a walk of a person.

### BACKGROUND ART

Patent Literature 1 discloses a technology of causing a self-propelling running device to: record an image captured by an image capturing device and information about an obstacle detected by an obstacle detector together with date and time information and position information; acquire a movement route per resident; and detect the obstacle having a possibility of hindering a walk of each resident around the acquired movement route per resident.

However, Patent Literature 1 fails to detect danger about a walk of a person in consideration of a relation between a walking ability of the person and a height of a step from a floor, and thus needs further improvement.

### CITATION LIST

### Patent Literature

Patent Literature 1: Japanese Patent Publication No. 6539845

### SUMMARY OF INVENTION

This disclosure has been achieved to solve the drawbacks described above, and has an object of providing a technology for appropriate walk assist in accordance with a walking ability of a person and a height of a step from a floor.

An information processing method according to an aspect of the disclosure, by a processor included in an information processing device, includes: acquiring, on the basis of sensing data about a person, lifting information indicating a lifting state of a leg of the person; acquiring height information indicating a height of a step from a floor in a space where the person moves; determining, on the basis of the lifting information and the height information, a dangerous level of a walk of the person; generating walk assist information in accordance with the dangerous level; and outputting the walk assist information.

This disclosure enables appropriate walk assist in accordance with a walking ability of a person and a height of a step from a floor.

### BRIEF DESCRIPTION OF DRAWINGS

FIG. 1 is a block diagram showing an example of a configuration of a server in a first embodiment of the disclosure.

FIG. 2 shows an example of a data configuration of action information.

FIG. 3 shows an example of a data configuration of an action pattern database.

FIG. 4 shows an example of a data configuration of environmental information.

FIG. 5 shows an example of a data configuration of an environment pattern database.

FIG. 6 shows an example of a data configuration of a dangerous action database.

FIG. 7 shows an example of a data configuration of a dangerous environment database.

FIG. 8 is a flowchart showing an example of a process by a server in the first embodiment of the disclosure.

FIG. 9 shows an example of a notification screen image displayed on a display of a terminal in the first embodiment.

FIG. 10 is a flowchart showing details of step S11 (dangerous level determination) in FIG. 8.

FIG. 11 is a block diagram showing an example of a configuration of a server in a second embodiment of the disclosure.

FIG. 12 is a flowchart showing an example of determining a notification time by the server in the second embodiment of the disclosure.

FIG. 13 is a flowchart showing an example of transmitting notification information by the server in the second embodiment of the disclosure.

FIG. 14 shows an example of a notification screen image displayed on a display of a terminal in the second embodiment of the disclosure.

FIG. 15 is a block diagram showing an example of a configuration of a server in a third embodiment of the disclosure.

FIG. 16 is a flowchart showing an example of a process by the server in the third embodiment of the disclosure.

FIG. 17 shows an example of a display screen image of a remodel proposal.

FIG. 18 is a block diagram showing an example of a configuration of a server in a fourth embodiment of the disclosure.

FIG. 19 shows an example of a notification screen image of training information.

### DESCRIPTION OF EMBODIMENTS

Knowledge forming the basis of the present disclosure Assuming a 100-year life, a walking ability would decrease due to aging or an injury, and an incident or accident, such as falling-over in a house, would be highly likely to occur. This results in increasing a risk of a shorter healthy life expectancy of a person. A resident tends to lead a daily life without noticing danger of falling-over in a situation where a sudden incident is likely to occur because of a decrease in a walking ability of the resident and a locational environmental factor. Once an incident occurs, the incident would cause a fracture leading to a serious health problem of being bedridden. Under the circumstances, a countermeasure needs to be taken in advance of an occurrence of such an incident.

To achieve the countermeasure, walk assist considering a relation between a walking ability of a person and a height of a step from a floor is desired. Patent Literature 1 merely detects an obstacle located around a movement route of a person and hindering a walk thereof, and fails to attain appropriate walk assist due to the lack of consideration of the relation between the walking ability of the person and the height of the step from the floor.

This disclosure has been achieved to solve the drawbacks described above.

An information processing method according to an aspect of the disclosure, by a processor included in an information processing device, includes: acquiring, on the basis of sensing data about a person, lifting information indicating a lifting state of a leg of the person; acquiring height information indicating a height of a step from a floor in a space where the person moves; determining, on the basis of the lifting information and the height information, a dangerous level of a walk of the person; generating walk assist information in accordance with the dangerous level; and outputting the walk assist information.

According to this configuration, the lifting information indicating the lifting state of the leg of the person is acquired, the dangerous level of the walk of the person is determined on the basis of the acquired lifting information and height information indicating the height of the step from the floor, and the walk assist information according to the dangerous level is output. This enables appropriate walk assist in accordance with the walking ability of the person and the height of the step from the floor.

The information processing method may further include: acquiring action pattern information indicating an action pattern of the person in the space to store the action pattern information in a memory. In the determining of the dangerous level, when it is determined that the dangerous level is equal to or higher than a threshold, dangerous action information may be generated by associating the dangerous level with the action pattern information including an action of the person and a place at a time of the determination of danger. In the generating of the walk assist information, notification information for giving notification about the action and the place at the time may be generated as the walk assist information on the basis of the dangerous action information. In the outputting of the walk assist information, the notification information may be presented.

This configuration gives notification about the action and the place which are likely to cause the falling-over, and thus allows a user to grasp such a place and such an action as to be highly likely to cause the falling-over.

In the information processing method, the action at the time may include a prior action made just prior to the time; and the notification information may include the prior action.

This configuration gives notification about the prior action which is likely to cause the falling-over, and thus allows the user to grasp such an action as to be highly likely to cause the falling-over.

The information processing method may further include: acquiring environmental pattern information indicating an environmental change pattern in the space and storing the environment pattern information in the memory. In the determining of the dangerous level, when it is determined that the dangerous level is equal to or higher than a threshold, dangerous environmental information may be generated by associating the dangerous level with the environment pattern information at the time of the determination of danger. In the generating of the walk assist information, notification information for giving notification about an environment where falling-over is likely to occur may be generated on the basis of the dangerous environmental information. In the outputting of the walk assist information, the notification information may be presented.

This configuration gives notification about an environment where the falling-over is likely to occur, and thus allows the user to grasp an environment where the falling-over is highly likely to occur.

The information processing method may further include: estimating whether there is a high likelihood of falling-over on the basis of at least one of dangerous action information associating action pattern information and the dangerous level with each other and the dangerous environmental information associating the environment pattern information and the dangerous level with each other. In the outputting of the walk assist information, the notification information may be presented in estimation that there is a high likelihood of the falling-over.

This configuration estimates whether there is a high likelihood of falling-over on the basis of at least one of the dangerous action information and the dangerous environmental information, and presents the notification information in estimation that there is the high likelihood of the falling-over, and therefore can alert the user by notifying the user of at least one of the dangerous action and the dangerous environment concerning the likelihood.

In the information processing method, the dangerous level may include a frequency of events that the height of the step indicated by the height information is determined to be equal to or larger than a leg lifting range indicated by the lifting information.

This configuration adopts, as the dangerous level, the frequency of the events that the height of the step is determined to be equal to or larger than the leg lifting range, and thus achieves prevention of excessive outputs of the walk assist information.

In the information processing method, in the outputting of the walk assist information, the walk assist information may be output at a time when the dangerous level is determined to be equal to or higher than a threshold.

According to this configuration, the walk assist information is output at the time when the dangerous level is determined to be the threshold, and therefore, the walk assist information can be output in real time, i.e., at the time when the falling-over would be highly likely to occur.

In the information processing method, the height information may include a position of the step with respect to the floor. In the acquiring of the lifting information, the acquired lifting information may be stored in a memory. The information processing method may further include: estimating, on the basis of a history of the lifting information stored in the memory, a prospective lifting of the leg in a future walk of the person; and specifying, on the basis of the prospective lifting of the leg and the height information, a step which is likely to cause falling-over in future.

This configuration attains specifying of a step which is likely to cause falling-over in future in consideration of the walking ability of the person decreasing in accordance with aging.

The information processing method may further include: generating a remodel proposal for the space to remove the likelihood of falling-over at the position of the specified step in future; and outputting the remodel proposal.

This configuration presents the remodel proposal to remove the likelihood of falling-over at the step which would be highly likely to cause the falling-over in future, and thus can encourage the remodel to remove the likelihood of the falling-over.

The information processing method may further include: presenting training information to improve a walking ability of the person in accordance with the dangerous level; and outputting the training information.

This configuration presents training information to improve the walking ability of the person in accordance with the dangerous level, and therefore succeeds in encouraging the person to execute training for the improvement of the walking ability.

In the information processing method, the training information may include a training place specified on the basis of the height information and located in the space.

According to this configuration, the training information includes the training place located in the space, and thus, the configuration can more reliably encourage the person to execute the training to improve the walking ability of the person.

In the information processing method, in the presenting of the training information, the training information may be presented when the dangerous level is determined to be equal to or higher than a threshold.

This configuration presents the training information when the dangerous level is determined to be equal to or higher than the threshold, and thus succeeds in presenting the training information to the person having a low walking ability.

An information processing device according to another aspect of the disclosure includes a processor, the processor executing: acquiring, on the basis of sensing data about a person, lifting information indicating a lifting state of a leg of the person; acquiring height information indicating a height of a step from a floor in a space where the person moves; determining, on the basis of the lifting information and the height information, a dangerous level of a walk of the person; generating walk assist information in accordance with the dangerous level; and outputting the walk assist information.

With this configuration, it is possible to provide an information processing device that exerts operational effects equivalent to those of the information processing method described above.

An information processing program according to further another aspect of the disclosure is an information processing program causing a computer to serve as an information processing device, by a processor, including: acquiring, on the basis of sensing data about a person, lifting information indicating a lifting state of a leg of the person; acquiring height information indicating a height of a step from a floor in a space where the person moves; determining, on the basis of the lifting information and the height information, a dangerous level of a walk of the person; generating walk assist information in accordance with the dangerous level; and outputting the walk assist information.

With this configuration, it is possible to provide an information processing program that exerts operational effects equivalent to those of the information processing method described above.

This disclosure can be realized as an information processing system caused to operate by the information processing program as well. Additionally, it goes without saying that the computer program is distributable as a non-transitory computer readable storage medium like a CD-ROM, or distributable via a communication network like the Internet.

Each of the embodiments which will be described below represents a specific example of the disclosure. Numeric values, shapes, constituent elements, steps, and the order of the steps described below in each embodiment are mere examples, and thus should not be construed to delimit the disclosure. Moreover, constituent elements which are not recited in the independent claims each showing the broadest concept among the constituent elements in the embodiments are described as selectable constituent elements. The respective contents are combinable with each other in all the embodiments.

### First Embodiment

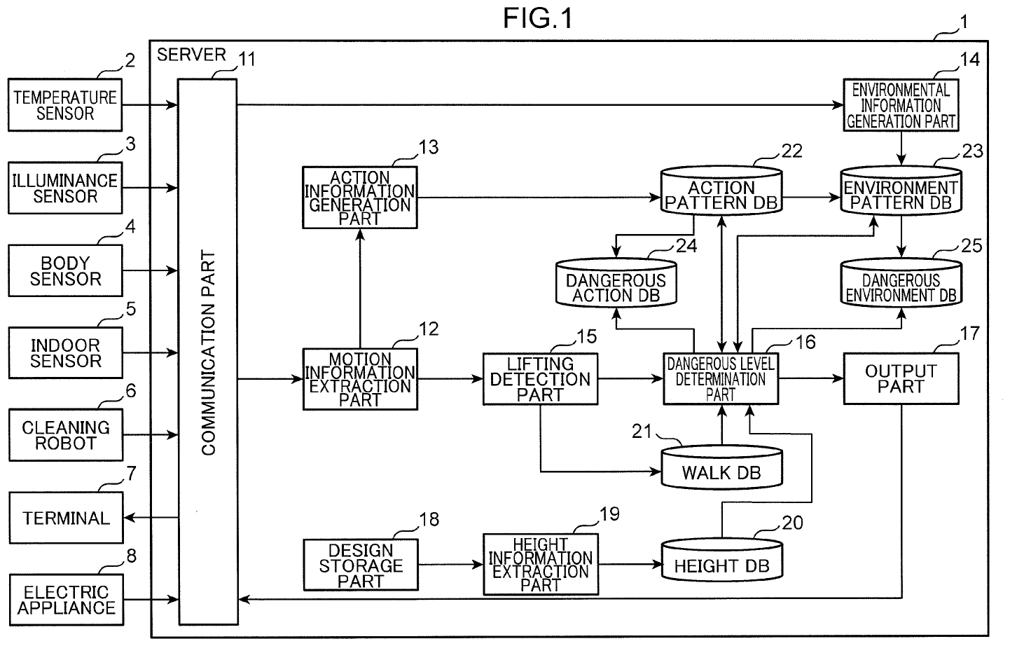

FIG. 1 is a block diagram showing an example of a configuration of a server 1 in a first embodiment of the disclosure. The server 1 is an example of the information processing device. The server 1 is connected to a temperature sensor 2, an illuminance sensor 3, a body sensor 4, an indoor sensor 5, a cleaning robot 6, a terminal 7, and an electric appliance 8 via, for example, a network. The network includes, for example, a wide area network having a mobile phone communication network and an internet communication network. The server 1 includes, for example, a cloud server.

The temperature sensor 2, the illuminance sensor 3, the indoor sensor 5, the cleaning robot 6, and the electric appliance 8 are arranged in a house of a user. The house of the user is an example of a space. The user is an example of a person. The temperature sensor 2 is located in one or more places in the house to measure a room temperature of each place, and transmits sensing data indicating the measured temperature of each place to the server 1 in a predetermined sampling period. The illuminance sensor 3 is located in one or more places in the house to measure illuminance of each place, and transmits sensing data indicating the measured illuminance of each place to the server 1 in a predetermined sampling period. The sensing data transmitted from the temperature sensor 2 and the illuminance sensor 3 includes, for example, a house ID indicating the house having been sensed, a place ID indicating the place having been sensed, a sensing time, and a sensor value.

The body sensor 4 includes, for example, an acceleration sensor or a gyro sensor attached to a leg of the user, and transmits sensing data indicating a motion of the leg of the user to the server 1 in a predetermined sampling period. The sensing data sent from the body sensor 4 includes, for example, a user ID of the user wearing or carrying the body sensor 4, a sensing time, and a sensor value. The body sensor 4 may be a smartwatch or a smartphone. When the body sensor 4 is in the form of the smartphone, the body sensor 4 may be put in a pocket of trousers or pants, or the like of the user.

The indoor sensor 5 includes, for example, an image sensor located in each of a plurality of places (e.g., on a ceiling) in the house of the user, and sends image data indicating movement of the user as sensing data to the server 1 in a predetermined sampling period. The cleaning robot 6 is a self-propelling robot for cleaning in the house of the user, captures an image of the user in the house by using an image sensor, and transmits image data based on the captured image of the movement of the user as sensing data to the server 1 in a predetermined sampling period. The sensing data from the image sensor includes, for example, the house ID, the place ID, a sensing time, and a sensor value. It is noted here that each of the indoor sensor 5 and the cleaning robot 6 may include a distance measurement sensor, in place of the image sensor, for capturing a distance image. Examples of the distance measurement sensor include a LiDAR and a laser range finder.

The terminal 7 includes, for example, an information terminal, such as a mobile information terminal and a tablet-type computer, and is carried by the user. The terminal 7 receives notification information for giving notification about a dangerous level of a walk from the server 1, and displays the received notification information on a display thereof.

Examples of the electric appliance 8 include a domestic electric appliance, such as a microwave oven, a water heater, a refrigerator, a washing machine, a television, and a cooker. The electric appliance 8 transmits an operational log to the server 1 in a predetermined sampling period.

The server 1 includes a communication part 11, a motion information extraction part 12, an action information generation part 13, an environmental information generation part 14, a lifting detection part 15, a dangerous level determination part 16, an output part 17, a design storage part 18, a height information extraction part 19, a height database (DB) 20, a walk database (DB) 21, an action pattern database (DB) 22, an environment pattern database (DB) 23, a dangerous action database (DB) 24, and a dangerous environment database (DB) 25. In FIG. 1, each of the motion information extraction part 12 to the height information extraction part 19 come into effect when the processor executes an information processing program. However, this is a mere example, and each of the motion information extraction part 12 to the height information extraction part 19 may be established in the form of a dedicated hardware circuit like an ASIC. Each of the walk database 21 to the dangerous environment database 25 includes a rewritable non-volatile storage device, such as a hard disk drive and a solid state drive.

The communication part 11 includes a communication circuit connecting the server 1 to the network. The communication part 11 inputs the sensing data transmitted from the temperature sensor 2 and the illuminance sensor 3 to the environmental information generation part 14. The communication part 11 inputs the sensing data transmitted from the body sensor 4, the indoor sensor 5, and the cleaning robot 6 to the motion information extraction part 12. The communication part 11 transmits the notification information generated by the output part 17 to the terminal 7.

The motion information extraction part 12 extracts motion information indicating a motion of a body of the user by analyzing the sensing data input from the communication part 11, and chronologically inputs the extracted motion information to the action information generation part 13 and the lifting detection part 15 in a predetermined sampling period.

The motion information extracted from the sensing data of the image sensor transmitted from the indoor sensor 5 and the cleaning robot 6 includes, for example, skeletal information about the user. The skeletal information includes information indicating connection among specific portions, such as a toe and an ankle, a distal end of an arm, a face, and a joint of the user linked via a leg, an arm, a neck, and a torso. The motion information extraction part 12 may extract the skeletal information by using, for example, a known skeletal detection algorithm like an open pose. The skeletal information includes the house ID, the place ID, and a sensing time contained in the sensing data at an extraction source. Besides, the motion information extraction part 12 may identify the user from the image data by using a face recognition technology, and cause the skeletal information to include the user ID of the identified user.

The motion information extracted from the sensing data of the acceleration sensor or the gyro sensor transmitted from the body sensor 4 includes, for example, leg height data indicating the height of the leg from the floor, the user ID, and a sensing time. The motion information extraction part 12 may calculate the leg height data by, for example, integrating the sensor value of the acceleration sensor or the gyro sensor. The leg height data includes the user ID and the sensing time included in the sensing data at the extraction source. The leg height data includes, for example, two-dimensional or three-dimensional coordinate data indicating a position of the height of the leg from the floor serving as a reference.

Hereinafter, described is a case where the server 1 manages one user in one house for convenience. Hence, the house ID and the user ID are excluded from the description. However, this is a mere example, and the server 1 may manage a plurality of houses and a plurality of users. In this case, each house may be identified by using a corresponding house ID, and each user may be identified by using a corresponding user ID.

The skeletal information is input to the action information generation part 13 and the lifting detection part 15, and the leg height data is input to the lifting detection part 15.

The action information generation part 13 generates action information indicating an action of the person by analyzing the motion information (skeletal information), and stores a history of the generated action information in an unillustrated memory.

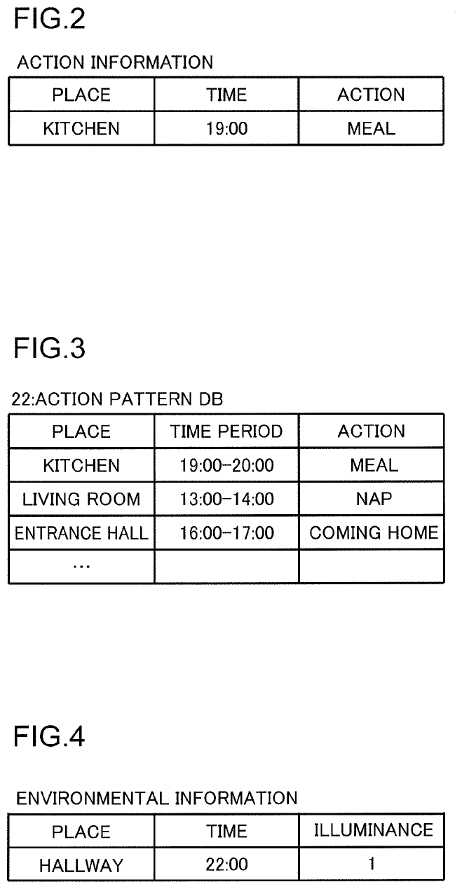

FIG. 2 shows an example of a data configuration of the action information. The action information includes a “place”, a “time”, and an “action”. The “place” represents a place where the user takes an action. The place is specified by the place ID included in the skeletal information. The “time” represents a sensing time when the action is taken. The “time” is specified from the sensing time included in the skeletal information. The “action” represents an action resulting from analyzing the skeletal information. Examples of the analyzed action include an action taken by the user in a daily life, such as, taking a meal, moving, coming home, taking a bath, and exercise. The action information generation part 13 may specify the action of the user from the skeletal information by employing, for example, a pattern matching way, or may specify the action of the user by using a learned model for estimating the action of the user from the skeletal information. The action information generation part 13 may estimate the action of the user by further using the operational log transmitted from the electric appliance 8 in addition to the skeletal information, may estimate the action of the user from the image data of the image sensor, or may estimate the action of the user from the information from the body sensor. The generated action information is stored in the action pattern database 22.

The action information generation part 13 generates action pattern information indicating an action pattern of the user from the generated action information, and stores the generated action pattern information in the action pattern database 22.

FIG. 3 shows an example of a data configuration of the action pattern database 22. The action pattern database 22 stores action pattern information including a “place”, a “time period”, and an “action. The action pattern information includes information indicating a certain action taken by the user in a certain place in a certain time period on one day. The “place” represents a place where the user takes an action. The “time period” represents a time period in which the user takes the action. The “action” represents the action taken by the user.

The action information generation part 13 may classify, for example, a history of the action information for each place and each action, and may generate action pattern information by specifying a time period in which the user takes the classified action in the classified place from the history of the classified action information.

The first row of the action pattern database 22 has storage of action pattern information indicating that the user has an action pattern of taking a meal in the kitchen or dining room in a time period from 19:00 to 20:00.

Referring back to FIG. 1, the environmental information generation part 14 generates environmental information indicating an environment in the house by analyzing the sensing data (sensing data of each of the temperature sensor 2 and the illuminance sensor 3) input from the communication part 11, and stores a history of the generated environmental information in the memory. FIG. 4 shows an example of a data configuration of the environmental information. The environmental information includes a “place”, a “time”, and “illuminance”. The “place” represents a sensing place of the illuminance, and is specified by the place ID included in the sensing data. The “time” represents a sensing time, and is specified from the sensing time included in the sensing data. The “illuminance” represents illuminance of the sensing place. In this example, the illuminance takes numeric values “1” to “5” in five stages. The numeric value “1” indicates the darkest illuminance, and the numeric value “5” indicates the brightest illuminance. The environmental information may include a temperature of the sensing place in addition to the illuminance. The example in FIG. 4 shows environmental information indicating the illuminance “1” of a hallway at 22:00.

The environmental information generation part 14 generates environment pattern information indicating an environmental change pattern of the house on one day from the history of the environmental information, and stores the environment pattern information in the environment pattern database 23.

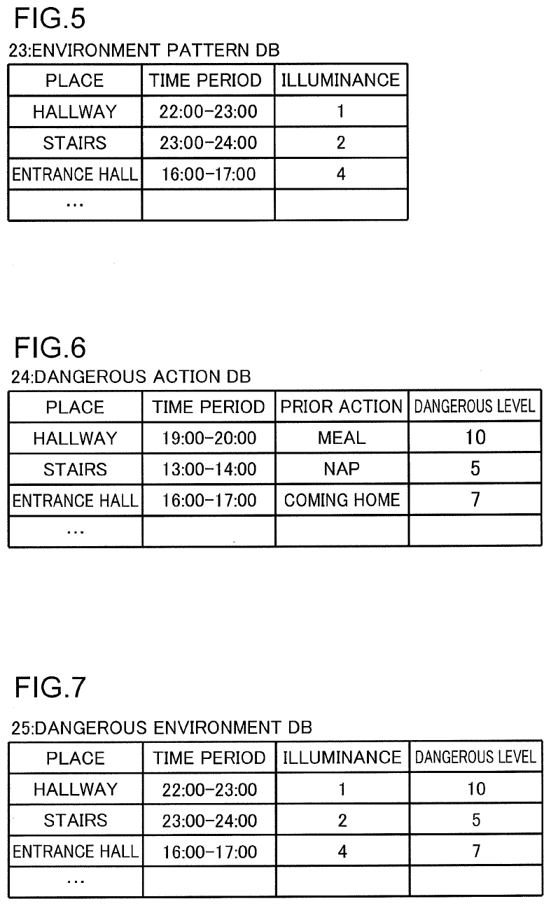

FIG. 5 shows an example of a data configuration of the environment pattern database 23. The environment pattern database 23 stores environment pattern information including a “place”, a “time period”, and “illuminance”. The environmental information generation part 14 may classify, for example, the history of the environmental information for each place and each illuminance, and may generate action pattern information by specifying a time period in which the classified illuminance is seen in the classified place from the history of the classified environmental information.

The first row of the environment pattern database 23 has storage of environment pattern information indicating that the illuminance of the hallway indicates “1” in a time period from 22:00 to 23:00.

Referring back to FIG. 1, the lifting detection part 15 generates, on the basis of the motion information (skeletal information and the leg height data) input from the motion information extraction part 12, lifting information indicating a lifting state of the leg of the user. The lifting information represents chronological data of a leg lifting range. The lifting information includes a place ID and a sensing time. The leg lifting range represents a maximum value of a vertical distance between the floor surface and a lowest position (e.g., a toe) of the leg in one walk cycle. When, a difference is seen between a leg lifting range of the left leg and a leg lifting range of the right leg, the smaller leg lifting range is adopted. The lifting detection part 15 may calculate the leg lifting range per walk cycle by using, for example, either the input leg height data or the input skeletal information, or may calculate the leg lifting range by using both the leg height data and the skeletal information. Alternatively, the lifting detection part 15 may calculate the leg lifting range per walk cycle by basically using the leg height data, and may calculate the leg lifting range per walk cycle by using the skeletal information when the leg lifting range is uncalculatable from the leg height data. From these perspectives, the lifting detection part 15 may calculate the leg lifting range per walk cycle by interpolating one of the skeletal information and the leg height data with the other. The generated lifting information is input to the dangerous level determination part 16 and stored in the walk database 21.

The dangerous level determination part 16 acquires the lifting information from the lifting detection part 15 and the walk database 21, acquires floor height information from the height database 20, and determines a dangerous level of a walk of the user on the basis of the acquired lifting information and the acquired height information. For instance, the dangerous level determination part 16 may calculate, as the dangerous level, a frequency of (the number of) events that a height of a step in a place indicated by a place ID included in the lifting information input from the lifting detection part 15 is determined to be equal to or larger than a leg lifting range indicated by the lifting information. The execution by the dangerous level determination part 16 will be described in detail later. Alternatively, the dangerous level may represent a proportion of the number of successes in passing through a certain step without stumbling to the total number of events of passing through the step. The dangerous level determination part 16 may determine that the user could pass through the step without stumbling when, for example, the leg lifting range is larger than the height of the step.

The dangerous level determination part 16 acquires, from the action pattern database 22, action pattern information related to the calculated dangerous level when the calculated dangerous level is equal to or higher than a threshold, generates dangerous action information by associating the acquired action pattern information and the calculated dangerous level with each other, and stores the generated dangerous action information in the dangerous action database 24.

FIG. 6 shows an example of a data configuration of the dangerous action database 24. The dangerous action database 24 stores the dangerous action information. The dangerous action information includes information about a place and a time period where and in which the user is highly likely to fall over. Specifically, the dangerous action information includes a “place”, a “time period”, a “prior action”, and a “dangerous level”. The “place” represents a place where the dangerous level is determined to be equal to or higher than the threshold. The “time period” represents a time period in which the “prior action” is taken. The “prior action” represents a prior action taken by the user just before the dangerous level determination part 16 determines that the dangerous level is equal to or higher than the threshold. The “dangerous level” represents a dangerous level determined to be equal to or higher than the threshold.

The dangerous level determination part 16 specifies action pattern information indicating the prior action with reference to the action pattern database 22 shown in FIG. 3 when the calculated dangerous level is equal to or higher than the threshold, and generates dangerous action information by associating the specified action pattern information, a current place where the user is, and the calculated dangerous level with one another, and stores the generated dangerous action information in the dangerous action database 24.

For instance, when determining that a dangerous level concerning the user being on the hallway at 20:02 is equal to or higher than the threshold, the dangerous level determination part 16 specifies taking a meal in the time period from “19:00 to 20:00” as a prior action from the action pattern database 22. Then, the dangerous level determination part 16 may generate dangerous action information by associating the current place “hallway” where the user is, the time period from “19:00 to 20:00” in which the prior action is taken, the prior action of taking the “meal”, and the calculated dangerous level of “10” with one another, and stores the generated dangerous action information in the dangerous action database 24. The dangerous level determination part 16 may specify the current place where the user is from action information corresponding to a current time.

The dangerous level determination part 16 acquires, from the environment pattern database 23, environment pattern information related to the calculated dangerous level when the calculated dangerous level is equal to or higher than the threshold, generates dangerous environmental information by associating the acquired environment pattern information and the calculated dangerous level with each other, and stores the generated dangerous environmental information in the dangerous environment database 25.

FIG. 7 shows an example of a data configuration of the dangerous environment database 25. The dangerous environment database 25 stores the dangerous environmental information. The dangerous environmental information includes information about a place and a time period where and in which the user is highly likely to fall over. Specifically, the dangerous environmental information includes a “place”, a “time period”, “illuminance”, and a “dangerous level”. The “place”, the “time period”, and the “illuminance” are the same as those in the environment pattern information shown in FIG. 5. The “dangerous level” represents a dangerous level determined to be equal to or higher than the threshold.

For instance, when determining that a dangerous level concerning the user being on the hallway at 22:10 is equal to or higher than the threshold, the dangerous level determination part 16 acquires, from the environment pattern database 23 shown in FIG. 5, relevant environment pattern information about the “hallway” in the time period from “22:00 to 23:00”, generates dangerous environmental information by associating the acquired environment pattern information with the calculated dangerous level “10”, and stores the generated dangerous environmental information in the dangerous environment database 25.

When the calculated dangerous level is equal to or higher than the threshold, the dangerous level determination part 16 generates walk assist information in accordance with the calculated dangerous level, and inputs the generated walk assist information to the output part 17. The walk assist information represents notification information for notifying the user of, for example, at least one of a dangerous action and a dangerous environment related to the walk of the user. For instance, when the calculated dangerous level is equal to or higher than the threshold, the dangerous level determination part 16 acquires a relevant dangerous action from the dangerous action database 24, acquires a relevant dangerous environment from the dangerous environment database 25, and generates, on the basis of the acquired dangerous action and dangerous environment, notification information for giving notification about the dangerous action and the dangerous environment. The relevant dangerous action is accompanied by the calculated dangerous level in connection with a time period and a place. The relevant dangerous environment is accompanied by the calculated dangerous level in connection with a time period and a place.

The walk database 21 chronologically stores the lifting information generated by the lifting detection part 15. For instance, the walk database 21 stores a place ID, a sensing time, and a leg lifting range in association with one another.

The design storage part 18 stores design data indicating a house structure including a floor plan for the user. The design data includes, for example, CAD data three-dimensionally showing the house structure.

The height information extraction part 19 extracts height information indicating a height of a step in each place from the design data stored in the design storage part 18. The height information extraction part 19 may acquire floor plan information about the house created by the cleaning robot 6 having employed the technology, such as the SLAM, and may extract the height information by using the acquired floor plan information.

The height database 20 stores the height information extracted by the height information extraction part 19. For instance, the height database 20 stores a place ID and a height of a step in association with each other. The place ID is an identifier specifying a collective space defining each section of the house, such as a hallway and a living room. When a single place has a plurality of steps, the height database 20 may store the height of each of the steps. The place ID may represent a coordinate indicating a specific position in the house.

The output part 17 transmits the notification information input from the dangerous level determination part 16 to the terminal 7 by using the communication part 11.

Heretofore, the configuration of the server 1 is described.

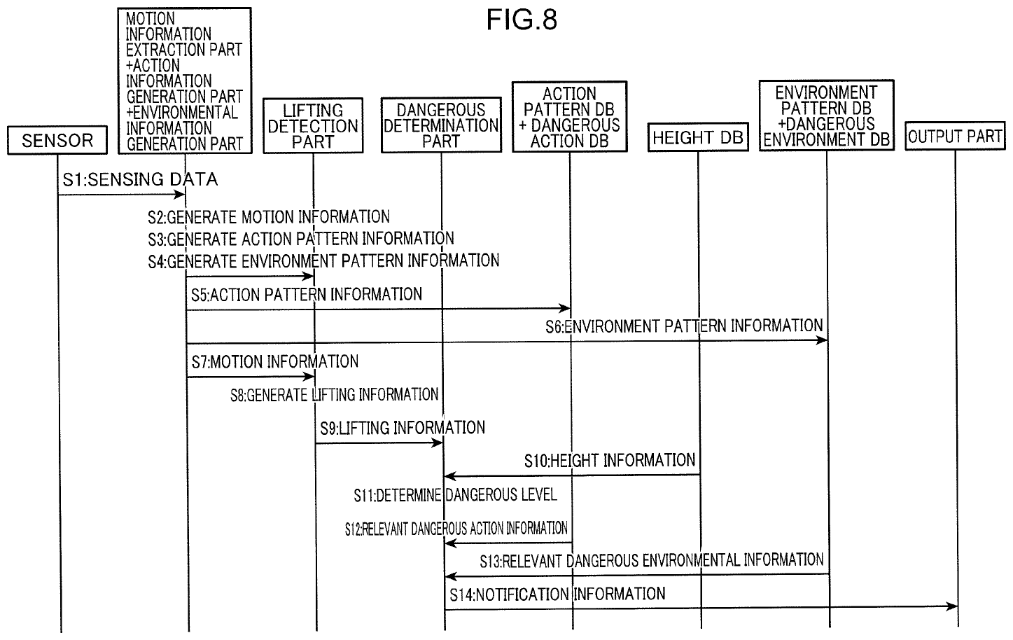

Next, a process by the server 1 will be described. FIG. 8 is a flowchart showing an example of the process by the server 1 in the first embodiment of the disclosure. The term “sensor” in FIG. 8 indicates the temperature sensor 2, the illuminance sensor 3, the body sensor 4, the indoor sensor 5, and the cleaning robot 6.

In step S1, the sensor transmits sensing data to the server 1. In step S2, the motion information extraction part 12 acquires sensing data of the image sensor transmitted from the indoor sensor 5 and the cleaning robot 6, acquires sensing data transmitted from the body sensor 4, and generates motion information by using the acquired sensing data.

In step S3, the action information generation part 13 generates action information from the motion information generated in step S2, and generates action pattern information from a history of the action information. In this manner, the action pattern information shown in FIG. 3 is generated. In step S4, the environmental information generation part 14 generates environmental information by using the sensing data transmitted from the temperature sensor 2 and the illuminance sensor 3 in step S1, and generates environment pattern information from a history of the environmental information. In this manner, the environment pattern information shown in FIG. 5 is generated.

In step S5, the action information generation part 13 stores, in the action pattern database 22, the action pattern information generated in step S3. In step S6, the environmental information generation part 14 stores, in the environment pattern database 23, the environment pattern information generated in step S4.

In step S7, the motion information extraction part 12 inputs the motion information (skeletal information and leg height data) generated in step S2 to the lifting detection part 15. In step S8, the lifting detection part 15 generates lifting information from the input motion information. In step S9, the lifting detection part 15 inputs the generated lifting information to the dangerous level determination part 16.

In step S10, the dangerous level determination part 16 acquires, from the height database 20, height information indicating a height of a step corresponding to a place ID included in the input lifting information.

In step S11, the dangerous level determination part 16 calculates a dangerous level on the basis of the lifting information acquired in step S9 and the height information acquired in step S10, and determines whether the calculated dangerous level is equal to or higher than a threshold. Here, the dangerous level is presumed to be equal to or higher than the threshold.

In step S12, the dangerous level determination part 16 acquires, from the dangerous action database 24, dangerous action information related to the calculated dangerous level.

In step S13, the dangerous level determination part 16 acquires, from the dangerous environment database 25, dangerous environmental information related to the calculated dangerous level.

In step S14, the dangerous level determination part 16 generates notification information on the basis of the relevant dangerous action information and dangerous environmental information, and inputs the generated notification information to the output part 17. Consequently, the output part 17 transmits the input notification information to the terminal 7. The terminal 7 generates a notification screen image from the received notification information and displays the generated notification screen image on a display thereof.

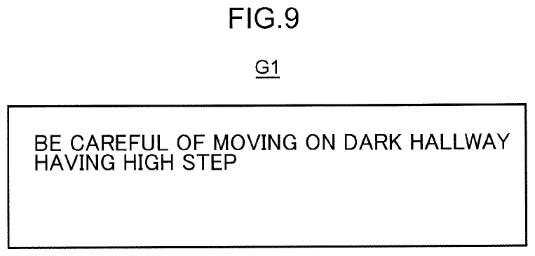

FIG. 9 shows an example of a notification screen image G1 displayed on the display of the terminal 7 in the first embodiment. The notification screen image G1 includes a message giving notification about a dangerous action (moving on a hallway), a dangerous environment (darkness of the hallway), and a dangerous factor (a high step). The notification allows the user to notice the step in moving on the hallway and avoid danger of falling-over in advance.

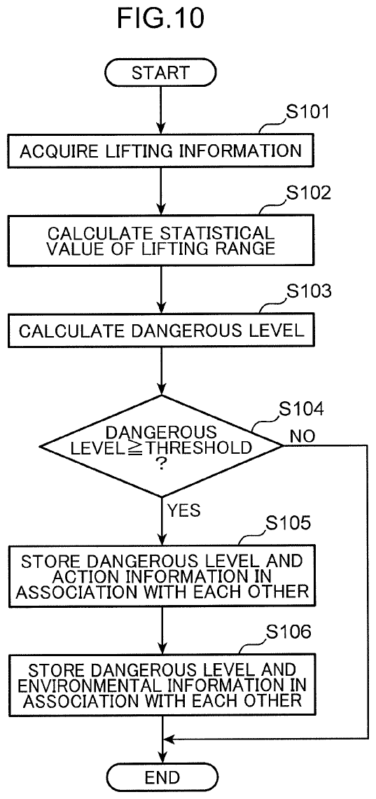

FIG. 10 is a flowchart showing details of step S11 (dangerous level determination) in FIG. 8. In step S101, the dangerous level determination part 16 acquires lifting information from the lifting detection part 15.

In step S102, the dangerous level determination part 16 acquires, from the walk database 21, a leg lifting range associated with a place ID included in the acquired lifting information, and calculates a statistical value of the acquired leg lifting range. Here, the dangerous level determination part 16 may calculate, as the statistical value of the leg lifting range, an average value of leg lifting ranges in a certain past period or an average weighting value of leg lifting ranges in the certain past period. A weight used for calculation of the average weighting value has, for example, a larger value for a newer leg lifting range. Alternatively, the dangerous level determination part 16 may calculate, as the statistical value of the leg lifting range, a minimum value of the leg lifting range in the certain past period, or may calculate, as the statistical value of the leg lifting range, a maximum value of the leg lifting range in the certain past period. Here, the dangerous level determination part 16 may calculate the statistical value of the leg lifting range by excluding the lifting range in a period in which a variance of the leg lifting range stored in the walk database 21 is equal to or larger than a given value.

In step S103, the dangerous level determination part 16 calculates, as the dangerous level, a frequency of events that the height of the step indicated by the place ID corresponding to the calculated statistical value of the leg lifting range is determined to be equal to or larger than the statistical value of the leg lifting range.

In step S104, the dangerous level determination part 16 determines whether the dangerous level is equal to or higher than a threshold. When determining that the dangerous level is equal to or higher than the threshold (YES in step S104), the dangerous level determination part 16 generates dangerous action information by associating the calculated dangerous level and action pattern information related to the dangerous level with each other, and stores the generated dangerous action information in the dangerous action database 24 (step 105). Contrarily, when the dangerous level falls below the threshold (NO in step S104), the process is finished without execution of steps S105 and S106. The threshold may be set to be smaller as a variance of the leg lifting range is larger. Owing to the setting, a determination criterion for the dangerous level is strictly settable when the variance is larger and the leg lifting range has a lower reliability.

In step S106, the dangerous level determination part 16 generates dangerous environmental information by associating the calculated dangerous level and environment pattern information related to the dangerous level with each other, and stores the generated dangerous environmental information in the dangerous environment database 25.

As described heretofore, according to the first embodiment, lifting information indicating a lifting state of a leg of a user is acquired, a dangerous level of a walk of the user is determined on the basis of the acquired lifting information and height information indicating a height of a step from a floor, and notification information according to the dangerous level is presented to the user. This enables appropriate walk assist in accordance with a walking ability of the user and the height of the step from the floor.

### Second Embodiment

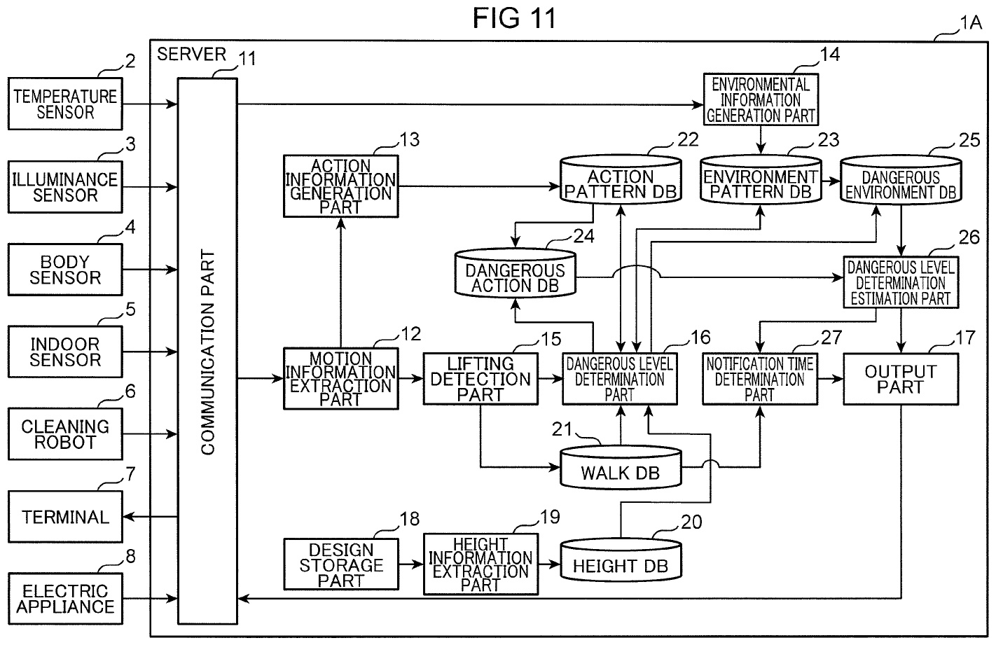

A second embodiment is aimed at presenting notification information to a user in a time period in which a dangerous level is equal to or higher than a threshold. FIG. 11 is a block diagram showing an example of a configuration of a server 1A in the second embodiment of the disclosure.

In the second embodiment, constituent elements which are the same as those in the first embodiment are given the same reference numerals and signs, and thus explanation therefor will be omitted.

The server 1A additionally includes a dangerous level determination estimation part 26 and a notification time determination part 27 in comparison with the server 1.

The dangerous level determination estimation part 26 estimates whether there is a high likelihood of falling-over on the basis of dangerous action information stored in a dangerous action database 24 and dangerous environmental information stored in a dangerous environment database 25.

Referring to FIG. 6, the first row of the dangerous action database 24 has storage of dangerous action information about danger of a walk of the user on a hallway after taking a meal in the time period from “19:00 to 20:00”. The dangerous level for the dangerous action information indicates “10” that is higher than a predetermined reference level, e.g., “6”, and thus, the user is highly likely to fall over. Hence, the dangerous level determination estimation part 26 estimates that there is a high likelihood of falling-over when the user moves on the hallway after taking the meal in the time period from “19:00 to 20:00”.

The second row of the dangerous action database 24 has storage of dangerous action information about danger of a walk of the user at stairs after taking a nap in the time period from “13:00 to 14:00”. The dangerous level for the dangerous action information indicates “5”, but the dangerous level is lower than the reference value, e.g., “6”. Hence, the dangerous level determination estimation part 26 estimates a low likelihood of falling-over in the movement on the stairs after the nap in the time period from “13:00 to 14:00”.

Referring to FIG. 7, the first row of the dangerous environment database 25 has storage of dangerous environmental information about danger in an environment of a hallway having illuminance “1” in a time period from “22:00 to 23:00”. The dangerous level for the dangerous action information indicates “10” that is higher than a predetermined reference level, e.g., “6”, and thus, the user is highly likely to fall over. The dangerous level for the dangerous environmental information indicates “10” that is higher than the reference level, e.g., “6”, and thus, the user is highly likely to fall over. Hence, the dangerous level determination estimation part 26 estimates that there is a high likelihood of falling-over in the environment indicated by the dangerous environmental information.

The second row of the dangerous environment database 25 has storage of dangerous environmental information about danger in an environment of stairs having illuminance “2” in a time period from “23:00 to 24:00”. The dangerous level concerning the user in the dangerous environmental information indicates “5” that is lower than the reference level, e.g., “6”. Hence, the dangerous level determination estimation part 26 estimates that there is a low likelihood of falling-over in the environment indicated by the dangerous environmental information.

Referring back to FIG. 11, the notification time determination part 27 determines a notification time for the notification information when the dangerous level determination estimation part 26 determines that there is a high likelihood of falling-over. The notification time represents, for example, a time when a situation where the user is agrees with a situation estimated to have the high likelihood of causing falling-over. Here, the dangerous level determination estimation part 26 may determine danger in consideration of past chronological action data in addition to data obtained just prior to a specific time and included in the dangerous environment database 25. For instance, in a case where a supper is taken in a time period from “21:00 to 22:00” prior to the time period from “22:00 to 23:00” and analysis of past data shows that a dangerous level around the stairs increases in the time period “22:00 to 23:00”, the dangerous level determination estimation part 26 may modify the dangerous level to be increased. Contrarily, in a case where a bath is taken in the time period from “21:00 to 22:00”, the dangerous level determination estimation part 26 may modify the dangerous level around the stairs to be lowered in the time period from “22:00 to 23:00”. A range of the analysis of past chronological data through tracing back may be determined in accordance with a processing time, a processing load, and analysis accuracy.

For instance, in the aforementioned example of the dangerous action information, moving on the hallway after taking the meal in the time period from “19:00 to 20:00” is estimated to lead to the high likelihood of falling-over. In this case, the notification time determination part 27 may determine, as the notification time, a time when the user enters the hallway in a predetermined period (e.g., ten minutes) continuous from the time “20:00”.

For instance, in the aforementioned example of the dangerous environmental information, the environment of the hallway having the illuminance “1” in the time period from “22:00 to 23:00” is estimated to have the high likelihood of causing falling-over. In this case, the notification time determination part 27 may determine, as the notification time, a time when the user enters the hallway in the time period from “22:00 to 23:00”. However, this is a mere example, and the notification time may be a time when the user is relaxing after taking the meal, such as watching a television.

Meanwhile, when determining that a leg lifting range of the user in a certain place decreases with reference to a walk database 21, the notification time determination part 27 may determine, as the notification time, a time when the user enters the certain place.

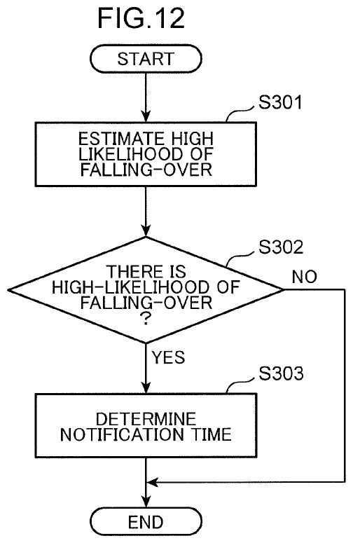

FIG. 12 is a flowchart showing an example of determining a notification time by the server 1A in the second embodiment of the disclosure. The determination is periodically executed at a predetermined interval, for example, once a day or once a week. In step S301, the dangerous level determination estimation part 26 estimates whether there is a high likelihood of falling-over by analyzing dangerous action information stored in the dangerous action database 24 and dangerous environmental information stored in the dangerous environment database 25.

When it is estimated that there is a high likelihood of falling-over (YES in step S302), the notification time determination part 27 determines a notification time (step S303). For instance, in the aforementioned example of the dangerous action information, a time when the user enters the hallway in a predetermined period continuous from the time period from “19:00 to 20:00” is determined as the notification time. By contrast, when it is determined that there is no high likelihood of the falling-over (NO in step S302), the flow is finished there.

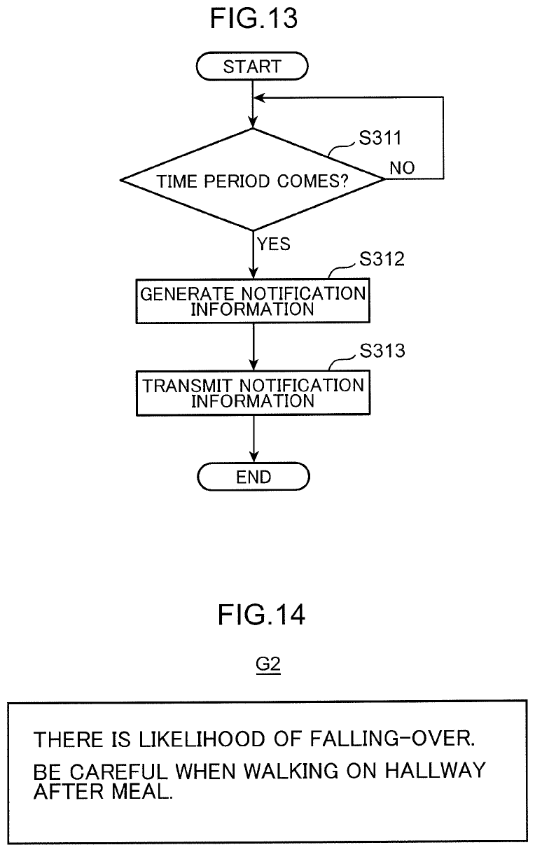

FIG. 13 is a flowchart showing an example of transmitting the notification information by the server 1A in the second embodiment of the disclosure. The steps in the flowchart are usually executed.

In step S311, the notification time determination part 27 monitors action information and determines whether a notification time comes on the basis of a monitoring result. For instance, when an action information generation part 13 generates action information satisfying a condition of each of a place and a time period defined with the notification time, the notification time determination part 27 determines that the notification time comes.

When the notification time comes (YES in step S311), the notification time determination part 27 generates notification information (step S312). Contrarily, when the notification time does not come (NO in step S311), the flow waits in step S311.

In step S313, an output part 17 transmits notification information to a terminal 7 by using a communication part 11.

FIG. 14 shows an example of a notification screen image G2 displayed on a display of the terminal 7 in the second embodiment of the disclosure. The notification screen image G2 includes a message showing a likelihood of falling-over. The notification screen image G2 further includes a message showing a likelihood of falling-over about a walk on a hallway after taking a meal. In this manner, the user is notified of the likelihood of falling-over through the notification screen image G2 in entering the hallway after taking the meal, and thus, the user can avoid falling over in advance. It is noted here that the notification may be given to the user by using a voice or vibration of a body sensor in place of the notification screen image.

As described heretofore, the server 1A in the second embodiment estimates whether there is a high likelihood of falling-over on the basis of at least one of dangerous action information and dangerous environmental information, and presents notification information in estimation that there is a high likelihood of falling-over, and therefore can alert the user by notifying the user of at least one of a dangerous action and a dangerous environment concerning the likelihood.

### Third Embodiment

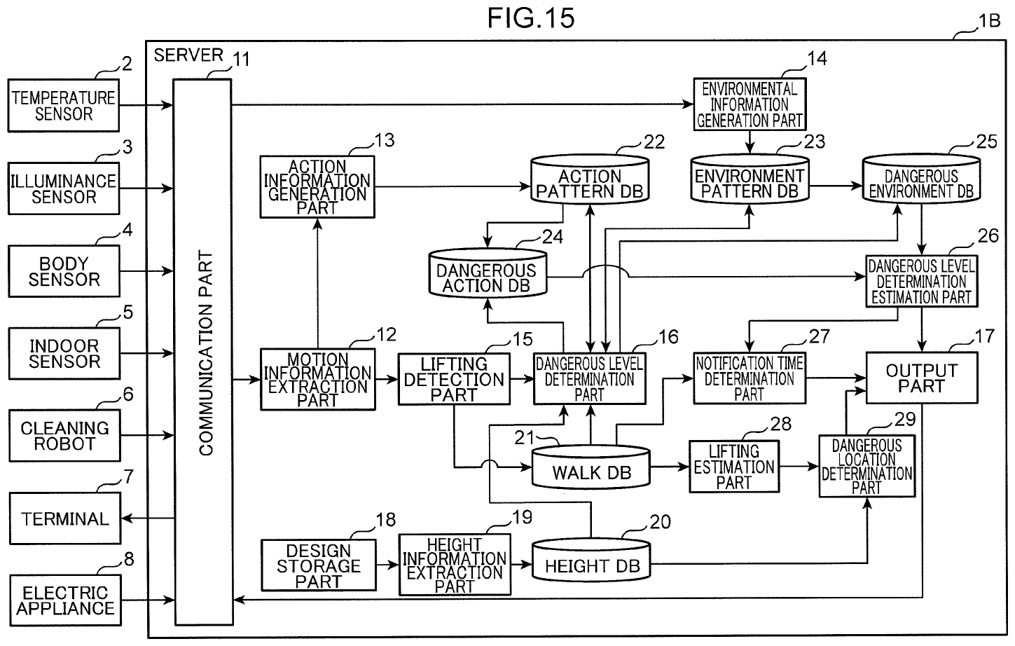

A third embodiment is aimed at specifying a certain place which is likely to cause falling-over of a user in future, and giving notification about a remodel proposal for the specified place. FIG. 15 is a block diagram showing an example of a configuration of a server 1B in the third embodiment of the disclosure. The server 1B additionally includes a lifting estimation part 28 and a dangerous location determination part 29 in comparison with the server 1A. In the embodiment, constituent elements which are the same as those in the first and second embodiments are given the same reference numerals, and thus explanation therefor will be omitted.

The lifting estimation part 28 estimates, on the basis of a history of lifting information stored in a walk database 21, a prospective lifting of a leg in a future walk of a person. The term “future” means a certain future time point, such as, after one year, three years, or ten years, but is not particularly limited thereto. For instance, the lifting estimation part 28 calculates an average shift value of a leg lifting range indicated by lifting information stored in the walk database 21 for each place, and estimates a prospective leg lifting range for each place from a time transition of the average shift value calculated for each place. For example, the lifting estimation part 28 may estimate the prospective leg lifting range by subjecting chronological data of the average shift value to linear interpolation. Adoptable examples of a time interval of the average shift value include an appropriate value, such as one day, one month, or one year.

Alternatively, the lifting estimation part 28 may estimate the prospective leg lifting range of the user in future by multiplying a current leg lifting range of the user by a decrease rate of the leg lifting range. For instance, the decrease rate is determined on the basis of a decay function defining a leg lifting in accordance with an age and obtained from a medical viewpoint. A decay rate is determined by inputting a current age of the user and a certain future time point to the decay function.

The dangerous location determination part 29 specifies a place which is likely to cause falling-over in future by comparing a leg lifting range in each place calculated by the lifting estimation part 28 and a height of a step in each place with each other. For example, when a prospective leg lifting range in a certain place (on a hallway) in future is lower than a step in the place (on the hallway), the step on the hallway is determined to be likely to cause falling-over in future.

The dangerous location determination part 29 generates a remodel proposal for removing the likelihood of falling-over in future at the step determined to be likely to cause the falling-over in future. The remodel proposal includes, for example, a message encouraging a remodel of lowering the step which is likely to cause falling-over in the place.

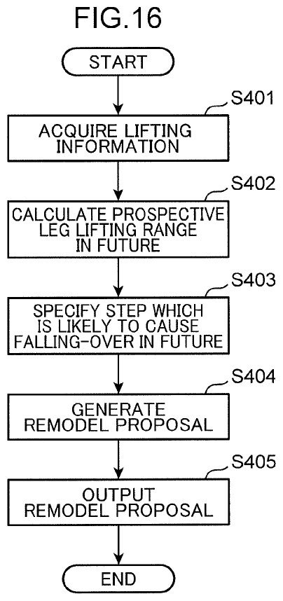

FIG. 16 is a flowchart showing an example of a process by the server 1B in the third embodiment of the disclosure. The steps in the flowchart are executed when, for example, a terminal 7 transmits a generation request for a remodel proposal to the server 1B. In step S401, the lifting estimation part 28 acquires a history of lifting information from the walk database 21.

In step S402, the lifting estimation part 28 calculates a prospective leg lifting range in each place by calculating an average shift value in each place about a leg lifting range indicated by the acquired history of lifting information and subjecting chronological data of the calculated average shift value to linear interpolation. Here, the future time point may take a value designated by the user and included in the generation request for the remodel proposal transmitted from the terminal 7.

In step S403, the dangerous location determination part 29 specifies a step which is likely to cause falling-over in future by comparing a prospective leg lifting range in each place and a height of a step in each place.

In step S404, the dangerous location determination part 29 generates a remodel proposal to remove the likelihood of falling-over at the specified step in future.

In step S405, an output part 17 transmits the generated remodel proposal to the terminal 7 by using a communication part 11. Consequently, the terminal 7 displays a notification screen image of the remodel proposal on a display thereof.

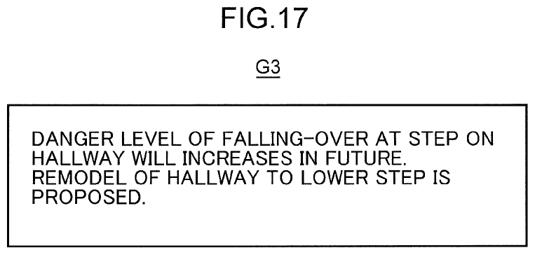

FIG. 17 shows an example of a notification screen image G3 of the remodel proposal. Here, a step on a hallway is specified as a step which is likely to cause falling-over in future, and thus, the notification screen image G3 includes a message showing danger of falling-over at the step on the hallway in future. The notification screen image G3 further includes a message encouraging a remodel to lower the step on the hallway.

Here, the notification screen image G3 shows the message of the remodel proposal, but may include an image of the remodel proposal. In an example of the image of the remodel proposal, a display object translucently showing a shape of a remodeled hallway excluding the step is superimposed on an image of the hallway in an overlooked view and displayed.

As described heretofore, the server 1B in the third embodiment specifies a step which is likely to cause falling-over in future in consideration of a walking ability of the user decreasing in accordance with aging, and generates a remodel proposal for removing the likelihood of falling over at the step, and thus can encourage the user to adopt the remodel to remove the likelihood of falling-over in future.

### Fourth Embodiment

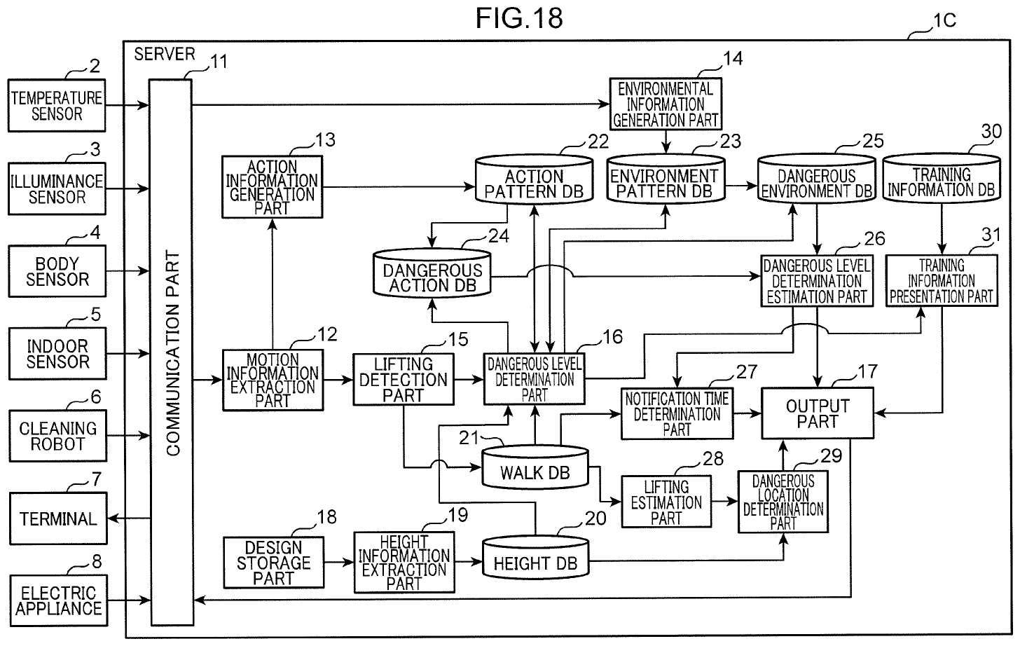

A fourth embodiment is aimed at generating training information to improve a walking ability of a person. FIG. 18 is a block diagram showing an example of a configuration of a server 1C in the fourth embodiment of the disclosure. In the embodiment, constituent elements which are the same as those in the first to third embodiments are given the same reference numerals and signs, and thus explanation therefor will be omitted.

The server 1C additionally includes a training information database (DB) 30 and a training information presentation part 31 in comparison with the server 1B. The training information database 30 stores, in advance, training information defining a training place in a house of a user and a training way in the training place. An example of the training place is a place including a step having an appropriate height for improving the walking ability in the house, e.g., stairs and an entrance hall.

The training information presentation part 31 presents the training information when a dangerous level determination part 16 determines that a dangerous level is equal to or higher than a threshold. Specifically, when the dangerous level determination part 16 determines that the dangerous level is equal to or higher than the threshold, the training information presentation part 31 acquires training information from the training information database 30 and inputs the acquired training information to an output part 17.

The training information presentation part 31 may present the training information at a time except for the time of the determination by the dangerous level determination part 16 that the dangerous level is equal to or higher than the threshold. The time may take a time period in which the user is relaxing. The training information presentation part 31 may determine that the user is relaxing by monitoring action information generated by an action information generation part 13. An example of the relaxing shows that the user is watching a television. The time for presenting the training information is not limited thereto, and may be, for example, a spare time from waking up to the breakfast, and is not particularly limited.

The output part 17 transmits the input training information to a terminal 7 by using a communication part 11. Consequently, the terminal 7 generates a notification screen image of the training information and displays the generated notification screen image on a display thereof.

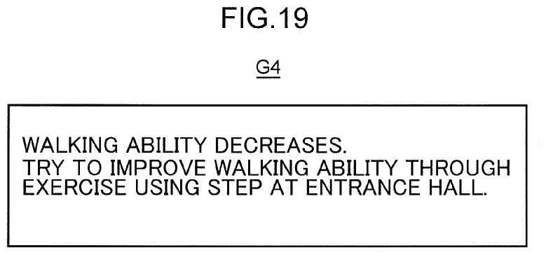

FIG. 19 shows an example of a notification screen image G4 of the training information. The notification screen image G4 includes a message encouraging an exercise to improve a walking ability in terms of a decrease in the walking ability. The notification screen image G4 further includes a message stating “by using a step at an entrance hall” to teach a place in the house for the exercise to improve the walking ability.

As described heretofore, the server 1C in the fourth embodiment presents training information to improve a walking ability of a user in accordance with a dangerous level, and accordingly, can encourage the user to execute training to improve the walking ability.

This disclosure can adopt modifications described below.
(1) Assist information is not limited to notification information, and may include a control signal from a walk assist suit worn by a user. A walk of the user wearing the walk assist suit is assisted by the walk assist suit when there is a high likelihood of falling-over.
(2) Lifting information about a leg includes a leg lifting range, but may further include a rightward or leftward deviation of the leg. The rightward or leftward deviation of the leg means an amount of motion of the leg in a left or right direction in a walk. The left or right direction perpendicularly intersects a forward direction and a vertical direction. The leftward or rightward deviation of the leg results in increasing the dangerous level, and accordingly, the dangerous level determination part 16 may modify the dangerous level to increase in accordance with an increase in the leftward or rightward deviation of the leg.

### INDUSTRIAL APPLICABILITY

The technology according to the disclosure is useful to prevent a person from falling over in a walk thereof.

1. An information processing method, by a processor included in an information processing device, comprising:
acquiring, on the basis of sensing data about a person, lifting information indicating a lifting state of a leg of the person;
acquiring height information indicating a height of a step from a floor in a space where the person moves;
determining, on the basis of the lifting information and the height information, a dangerous level of a walk of the person;
generating walk assist information in accordance with the dangerous level; and
outputting the walk assist information.

2. The information processing method according to claim 1, further comprising:
acquiring action pattern information indicating an action pattern of the person in the space to store the action pattern information in a memory, wherein:
in the determining of the dangerous level, when it is determined that the dangerous level is equal to or higher than a threshold, dangerous action information is generated by associating the dangerous level with the action pattern information including an action of the person and a place at a time of the determination of danger;
in the generating of the walk assist information, notification information for giving notification about the action and the place at the time is generated as the walk assist information on the basis of the dangerous action information; and
in the outputting of the walk assist information, the notification information is presented.

3. The information processing method according to claim 2, wherein the action at the time includes a prior action made just prior to the time, and
the notification information includes the prior action.

4. The information processing method according to claim 1, further comprising acquiring environmental pattern information indicating an environmental change pattern in the space and storing the environment pattern information in the memory, wherein:
in the determining of the dangerous level, when it is determined that the dangerous level is equal to or higher than a threshold, dangerous environmental information is generated by associating the dangerous level with the environment pattern information at the time of the determination of danger;
in the generating of the walk assist information, notification information for giving notification about an environment where falling-over is likely to occur is generated on the basis of the dangerous environmental information; and,
in the outputting of the walk assist information, the notification information is presented.

5. The information processing method according to claim 4, further comprising estimating whether there is a high likelihood of falling-over on the basis of at least one of dangerous action information associating action pattern information and the dangerous level with each other and the dangerous environmental information associating the environment pattern information and the dangerous level with each other, wherein,
in the outputting of the walk assist information, the notification information is presented in estimation that there is a high likelihood of the falling-over.

6. The information processing method according to claim 1, wherein the dangerous level includes a frequency of events that the height of the step indicated by the height information is determined to be equal to or larger than a leg lifting range indicated by the lifting information.

7. The information processing method according to claim 1, wherein, in the outputting of the walk assist information, the walk assist information is output at a time when the dangerous level is determined to be equal to or higher than a threshold.

8. The information processing method according to claim 1, wherein the height information includes a position of the step with respect to the floor, and,
in the acquiring of the lifting information, the acquired lifting information is stored in a memory, the information processing method further comprising:
estimating, on the basis of a history of the lifting information stored in the memory, a prospective lifting of the leg in a future walk of the person; and
specifying, on the basis of the prospective lifting of the leg and the height information, a step which is likely to cause falling-over in future.

9. The information processing method according to claim 8, further comprising:
generating a remodel proposal for the space to remove the likelihood of falling-over at the position of the specified step in future; and
outputting the remodel proposal.

10. The information processing method according to claim 1, further comprising:
presenting training information to improve a walking ability of the person in accordance with the dangerous level; and
outputting the training information.

11. The information processing method according to claim 10, wherein the training information includes a training place specified in advance on the basis of the height information and located in the space.

12. The information processing method according to claim 10, wherein, in the presenting of the training information, the training information is presented when the dangerous level is determined to be equal to or higher than a threshold.

13. An information processing device, comprising a processor which executes:
acquiring, on the basis of sensing data about a person, lifting information indicating a lifting state of a leg of the person;
acquiring height information indicating a height of a step from a floor in a space where the person moves;
determining, on the basis of the lifting information and the height information, a dangerous level of a walk of the person;
generating walk assist information in accordance with the dangerous level; and
outputting the walk assist information.

14. A non-transitory computer readable recording medium storing an information processing program causing a computer to serve as an information processing device, by a processor, comprising:
acquiring, on the basis of sensing data about a person, lifting information indicating a lifting state of a leg of the person;
acquiring height information indicating a height of a step from a floor in a space where the person moves;
determining, on the basis of the lifting information and the height information, a dangerous level of a walk of the person;
generating walk assist information in accordance with the dangerous level; and
outputting the walk assist information.
